(19) United States
(12) Patent Application Publication
(10) Pub. No.: US 2018/0221428 A1
(43) Pub. Date: Aug. 9, 2018

(54) Quit Your Itching Cream

(71) Applicant: Lisa Judson, Albany, OR (US)

(72) Inventor: Lisa Judson, Albany, OR (US)

(21) Appl. No.: 15/428,949

(22) Filed: Feb. 9, 2017

(51) Int. Cl. A61K 36/889; A61Q 19/00; A61K 36/185; A61K 36/534; A61K 36/53; A61K 8/92; A61K 36/61

(52) U.S. Cl. CPC A61K 36/889; A61Q 19/007; A61K 36/185; A61K 2800/5922; A61K 36/53; A61K 8/922; A61K 36/61; A61K 36/534

(57) ABSTRACT

The present invention relates to compositions and methods for the treatment of topical skin conditions, comprising of peppermint oil, lavender oil, tea tree oil and a suitable amount of a pharmaceutically acceptable carriers, shea butter and coconut oil.

### ORIGIN STORY

I was trying to do homework when my mother would not stop complaining about a cold sore on her lip. I became frustrated with her and went into the kitchen and combined various waxes with some essential oils and had her try the mixtures to see if they helped or not. After years of such experiments I came up with this mixture.

### PROPERTIES

Shea butter is commonly used for its cosmetic properties as a moisturizer, absorbing without feeling greasy. The properties include being anti-inflammatory and can help treat scars, eczema, burns, rashes, acne, dry skin, blemishes, dark spots, skin discolorations, chapped lips, wrinkles, stretchmarks, and in lessening psoriasis. Shea butter has a natural SPF varying between zero and six.

Coconut oil has antioxidant properties that help protect skin from free radical damage cause by sun exposure. Coconut oil is a poor oxidizer and largely shelf stable. Coconut oil is a saturated fat mostly medium chain fatty acids and is a triglyceride of lauric acid.

Peppermint oil is an essential oil, Menthaarvensis var. PiperascensCornmint and Japanese Mint and Mentha×piperita subsp. nothosubsp. Piperita Contain a large concentration of natural pesticides. The oil also has natural anti-inflammatory properties and can be soothing on dry skin.

Lavender oil is an essential oil from Lavandulalotifolia. It is obtained by distillation, is colorless and insoluble in water. This oil can be soothing to dry skin and has been used historically to repel pests. The oil has natural bleaching properties when applied to skin and exposed to sunlight and can cause light spots to occur.

Tea tree oil or Melaleuca alterrnifolia is derived from the leaves of the plant and used for a variety of skin conditions including dandruff, acne, lice, herpes and other skin infections. The oil is toxic when taken orally but has some promise as an antimicrobial when used in low concentrations in skin care products and cosmetics.

1. A composition effective for healing dry skin conditions, the composition comprising:
(a) shea butter in an amount from 49% to 66% by weight,
(b) coconut oil in an amount from 49% to 66% by weight
(c) peppermint oil in an amount from 0.0001% to 1% by weight
(d) lavender oil in an amount from 0.0001% to 1% by weight
(e) tea tree oil in an amount from 0.0001% to 1% by weight.

2. A composition effective for healing dry or itchy skin conditions.

3. Method of preparation of claim 1. Combine shea butter and coconut and heat till fluid. Mix and stir off heat till oils cool to 75 to 85 degrees fahrenheit. While stirring add peppermint oil, lavender oil, and tea tree oil and stir till combined. The resulting mixture should be pale yellow, and creamy to the touch.
